(19) United States
(12) Patent Application Publication
(10) Pub. No.: US 2020/0384283 A1
(43) Pub. Date: Dec. 10, 2020

(54) PHOTOBIOMODULATION THERAPY TO AMELIORATE MULTIPLE SCLEROSIS

(71) Applicant: MULTI RADIANCE MEDICAL, Solon, OH (US)

(72) Inventors: Douglas JOHNSON, Brownstown, MI (US); Max KANARSKY, Solon, OH (US)

(21) Appl. No.: 16/760,806

(22) PCT Filed: Oct. 30, 2018
(86) PCT No.: PCT/US2018/058106

§ 371 Date: Apr. 30, 2020

Related U.S. Application Data
(60) Provisional application No. 62/578,719, filed on Oct. 30, 2017.

(51) Int. Cl. A61N 2/06; A61N 5/06

(52) U.S. Cl. CPC A61N 2/06; A61N 5/0622; A61N 2005/0663; A61N 2005/067; A61N 2005/0644; A61N 2005/0652; A61N 2005/0659

(57) ABSTRACT

Photobiomedulation therapy (PBMT) can be applied to a skeletal muscle ameliorate multiple sclerosis (MS). A light source device can be contacted to a subjects skin proximal to the skeletal muscle of a patient affected by MS. A light signal (with one or more wavelengths from 400-1100 nm) can be applied in at least one of a pulsed operating mode, a continuous operating mode, and a super-pulsed operating mode through the light source device to the skeletal muscle. The light signal is applied for a time sufficient to stimulate a phototherapeutic response in the skeletal muscle and/or a nerve associated with the skeletal muscle. PBMT applied in this manner provides a noninvasive, safe and effective therapy to mitigate muscle weakness and/or muscle fatigue, improve muscle recovery, reduce general fatigue, and maintain muscle strength, thereby increasing quality of life of the patient affected by MS.

### CROSS-REFERENCE TO RELATED APPLICATIONS

This application claims the benefit of U.S. Provisional Application No. 62/578,719, filed Oct. 30, 2017, entitled “AMELIORATION OF MULTIPLE SCLEROSIS”. This provisional application is hereby incorporated by reference in its entirety for all purposes.

### TECHNICAL FIELD

The present disclosure relates generally to photobiomodulation therapy (PBMT) and, more specifically, to systems and methods that apply PBMT to a skeletal muscle of a patient affected by multiple sclerosis (MS) to ameliorate MS.

### BACKGROUND

Multiple Sclerosis (MS) is a chronic, progressive disease of the central nervous system (CNS) that affects approximately 2.5 million people worldwide. MS often manifests itself through severe fatigue. Such severe fatigue can have a detrimental effect on the quality of life of a patient with MS. This severe fatigue is due to muscle weakness and/or fatigue. Traditionally, this muscle weakness and fatigue has been thought to be caused by a deconditioning of muscles of patients with MS due to lack of activity. However, current exercise therapies are ineffective with regard to the muscle weakness and fatigue. It is now thought that the deconditioning may be due to neurodegeneration characteristic of MS.

With MS, myelin sheaths of axons of the brain, spinal cord and optic nerve become damaged, resulting in demyelination and scarring. Such demyelination and scaring leads to poor nerve conduction. Common physical therapy and weight training therapies are not effective to strengthen muscles that do not receive adequate nerve conduction. A noninvasive, safe and effective therapy for mitigating muscle fatigue, improving muscle recovery, and maintaining muscle strength would greatly increase the quality of life of patients with MS and other neurological diseases that manifest in severe muscle fatigue.

### SUMMARY

The present disclosure relates generally to photobiomodulation therapy (PBMT) and, more specifically, to systems and methods that apply PBMT to a skeletal muscle of a patient affected by multiple sclerosis (MS) to ameliorate MS. PBMT provides a noninvasive, safe and effective therapy to mitigate muscle fatigue, improve muscle recovery, and maintain muscle strength, thereby increasing quality of life of a patient with MS.

In one aspect, the present disclosure can include a method for applying PBMT to a skeletal muscle of a patient affected by MS to ameliorate MS. The method can include placing a light source device proximal to a skeletal muscle of a patient affected by MS and applying a light signal through the light source device to the skeletal muscle of the patient affected by MS. The light signal can be applied in at least one of a pulsed operating mode, a continuous operating mode, or a super-pulsed operating mode. The light signal can be applied for a time sufficient to stimulate a phototherapeutic response in the skeletal muscle of the patient affected by MS.

In another aspect, the present disclosure can include a light source device to apply PBMT to a skeletal muscle of a patient affected by MS to ameliorate MS. The light source device can be configured to be placed proximal to a muscle of a patient affected by MS. The light source device can include at least one light source configured to apply a light signal in at least one of a pulsed operating mode, a continuous operating mode, and a super-pulsed operating mode to the skeletal muscle of the patient affected by MS. The light source device can also include a processing unit preprogrammed with a time for application of the light signal, wherein the time is sufficient to stimulate a phototherapeutic response in the skeletal muscle of the patient affected by MS.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram illustration showing an example of a system that configures and applies photobiomodulation therapy (PBMT) to a skeletal muscle of a patient affected by multiple sclerosis (MS) to ameliorate MS in accordance with an aspect of the present disclosure;

FIG. 2 is a block diagram illustration showing an example configuration of light sources within the light delivery source cluster of FIG. 1;

FIG. 3 is a process flow diagram of an example method for applying PBMT to a skeletal muscle of a patient affected by MS to ameliorate MS in accordance with another aspect of the present disclosure;

FIG. 4 is a process flow diagram of another example method for applying PBMT to another skeletal muscle of the patient affected by MS to further ameliorate MS in accordance with a further aspect of the present disclosure;

FIG. 5 shows a diagram of an example of the light source device of FIG. 1;

FIG. 6 shows a picture of another example of the light source device of FIG. 1;

FIG. 7 shows a picture of an example use of the light source device shown in FIG. 6; and

FIG. 8 shows a picture of another example of the light source device of FIG. 1.

### DETAILED DESCRIPTION

### I. Definitions

Unless otherwise defined, all technical terms used herein have the same meaning as commonly understood by one of ordinary skill in the art to which the present disclosure pertains.

In the context of the present disclosure, the singular forms “a,” “an” and “the” can also include the plural forms, unless the context clearly indicates otherwise.

As used herein, the terms “comprises” and/or “comprising” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “multiple sclerosis (MS)” refers to an autoimmune disease in which the body's immune system attacks and destroys myelin in the central nervous system (CNS), leading to scarring and, eventually, deterioration or damage of the nerves themselves. MS causes communication problems both within the CNS and between the CNS and the rest of the body. Patients with MS often experience muscle weakness and/or muscle fatigue, which may contribute to symptomatic fatigue commonly reported by patients suffering from MS.

As used herein, the term “central nervous system (CNS)” refers to a division of the nervous system that integrates and coordinates the activities of the entire body. The CNS includes the brain, spinal cord, and optic nerves.

As used herein, the term “myelin” refers to the fatty substance that surrounds and insulates nerve fibers (also referred to as axons) to speed conduction in the nerve fibers.

As used herein, the term “ameliorate” refers to making something better or more tolerable.

As used herein, the term “photobiomodulation” refers to the application of a light signal to a portion of a subject's body to induce a phototherapeutic response in cells within the portion of the subject's body.

As used herein, the term “photobiomodulation therapy (PBMT)” refers to a drug-free, non-invasive treatment procedure, in which a light signal is applied to a certain region of a subject's body to treat a certain medical condition (e.g., pain, injury, disorder, disease, or the like) with a goal of ameliorating the certain medical condition via a phototherapeutic response. In some instances, PBMT can be used alone to induce a phototherapeutic response, but in other instances, PBMT can be used in combination with other therapies (e.g., a pharmaceutical therapy, exercise therapy, and the like).

As used herein, the term “phototherapeutic response” refers to a biological reaction to application of PBMT to a portion of the patient's body.

As used herein, the term “light signal” refers to light having at least one wavelength. However, the light signal may include a combination of lights having wavelengths that create a synergistic effect when combined and improves the percentage of available light at greater tissue depths. In some instances, the wavelengths can be within a wavelength range of 400-1100 nm. For example, the wavelengths can include at least one wavelength corresponding to the visible range of the electromagnetic spectrum (e.g., red light) and at least one wavelength corresponding to the near-infrared or infrared range of the electromagnetic spectrum.

As used herein, the term “light source device” refers to a mechanical implement that can deliver a light signal of PMBT to a portion of the subject's body. Examples of the light source device include a probe, a flexible array device, or the like.

As used herein, the term “light source” refers to a component of a light source device that delivers one or more lights of different wavelengths. For example, the light source can be a low-level laser source (e.g., a laser light emitting diode (LED)) that generates coherent light. The low-level laser source can operate in a super pulsed mode that generates ultrashort pulses with a high peak power and minimal heat. As another example, the light source can be an incoherent light source, such as a traditional LED or light bulb. The incoherent light source can operate in a pulsed mode and/or a continuous mode.

As used herein, the term “muscle” refers to skeletal muscle. A skeletal muscle, as used herein, can include a single muscle and/or a muscle group.

As used herein, the term “proximal” refers to a location that is near a target (e.g., skeletal muscle). For example, a device that is located proximal a muscle or muscle group can be located over the muscle or muscle group, but need not be directly over the center of the muscle or the muscle group.

As used herein, the term “sufficient” refers to an amount adequate enough to satisfy a condition. For example, “a time sufficient to stimulate a phototherapeutic response in skeletal muscle” can refer to a light signal being applied to a skeletal muscle for a time adequate enough to stimulate the phototherapeutic response in the skeletal muscle or an associated nerve.

As used herein, the term “direct” refers to the absence of intervening elements. For example, a device that directly contacts a skin surface has no intervening elements between the device and the skin surface. When the term “contact” is used herein, it means “direct contact” unless otherwise stated.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

### II. Overview

The present disclosure relates generally to photobiomodulation therapy (PBMT) and, more specifically, to systems and methods that apply PBMT to a skeletal muscle of a patient affected by multiple sclerosis (MS) to ameliorate MS. A common complaint of patients suffering from MS is general fatigue. These patients often experience muscle weakness and/or muscle fatigue, which may contribute to the reported general fatigue. Application of PBMT, according to certain therapy parameters, through skin proximal to the skeletal muscle, can stimulate a phototherapeutic response that leads to the mitigation of the muscle weakness and/or muscle fatigue and reduction in complaints of general fatigue. The PBMT provides a noninvasive, safe and effective therapy to mitigate muscle weakness and/or muscle fatigue, improve muscle recovery, reduce incidences of general fatigue, and maintain muscle strength, thereby increasing quality of life of the patient affected by MS. PBMT can be used as an independent therapy strategy. However, in some instances, a greater benefit may be seen if the PBMT is performed as an adjunct to existing therapeutic agents, including exercise.

### III. Photobiomodulation Therapy (PBMT)

Muscle weakness and/or muscle fatigue suffered by an MS patient may be due to neurodegeneration characteristic of MS, leading to decreased conduction between the central nervous system and peripheral nervous system. The impaired conduction associated with neurodegeneration can be due to a decreased mitochondrial oxidative capacity. The decreased mitochondrial oxidative capacity can improve due to modulation of mitochondrial cytochrome c-oxidase (CCO) (a photoacceptor), the phototherapeutic response triggered by the PBMT. Modulating CCO can lead to stopping or slowing the neurodegeneration characteristic of MS.

While not wishing to be bound by theory, there is strong evidence to suggest that one of the basic mechanisms of PBMT is the acceleration of electron transfer by electromagnetic radiation in the visible and near infrared region of the spectrum, via the modulation of CCO activity. Traditionally, PBMT has attempted to modulate CCO activity using a single wavelength in the visible or near infrared region of the spectrum. However, the use of such single wavelengths cannot effectively modulate CCO activity since the single wavelength is limited by its specific absorption spectrum. The light signal used herein has a combination of wavelengths, which are used concurrently, providing an overlapping effect of peak activation, which accelerates CCO activity. Additionally, the time of CCO activation is prolonged across the entire therapeutic window by delivering much smaller doses across many wavelengths, rather than a single wavelength of a greater power. The multiple wavelengths enhance adenosine triphosphate (ATP) production, requiring less energy, and provides continual photodissociation of nitric oxide (NO), not only from CCO, but also from intracellular stores like nitrosylated forms of hemoglobin and myoglobin. NO is a potent vasodilator and PBMT can increase the vasodilation due to NO and increases the availability of oxygen to treated cells, and allows for greater traffic of immune cells into tissue.

### IV. Systems

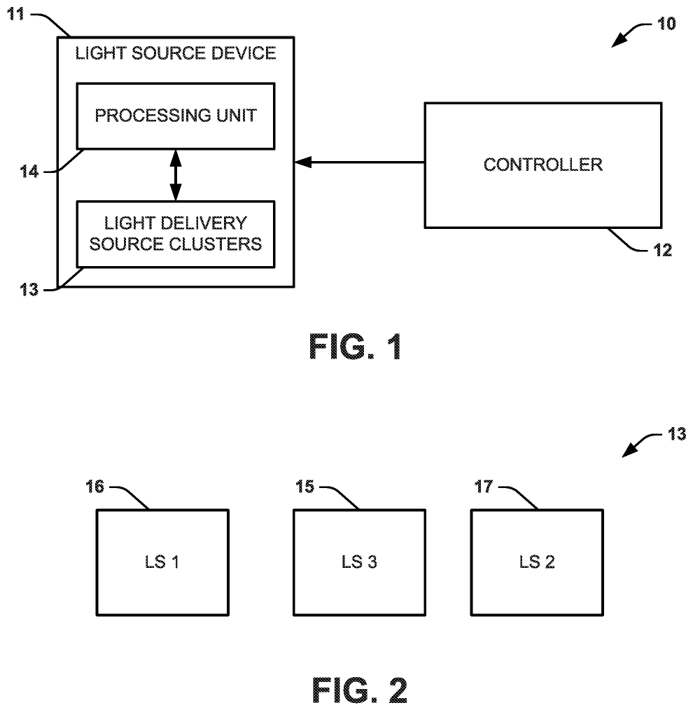

One aspect of the present disclosure can include a system 10 (FIG. 1) configures and applies photobiomodulation therapy (PBMT) to a skeletal muscle of a patient affected by multiple sclerosis (MS). The PBMT can cause a phototherapeutic response in the skeletal muscle and/or the nerves innervating the skeletal muscle, reducing neurodegeneration characteristic of MS. The phototherapeutic response can decrease neurodegeneration by improving mitochondrial oxidative capacity due to modulation of mitochondrial cytochrome c-oxidase (CCO). Accordingly, the PBMT provides a noninvasive, safe and effective therapy to mitigate muscle weakness and/or muscle fatigue, improve muscle recovery, reduce incidences of general fatigue, and maintain muscle strength, thereby increasing quality of life of the patient affected by MS. PBMT can be used as an independent therapy strategy for patients with MS. However, in some instances, a greater benefit may be seen if the PBMT is performed as an adjunct to existing therapeutic agents, including exercise, for patients with MS.

The system 10 can include at least a light source device 11 that delivers the PBMT to the skeletal muscle and a controller 12 to deliver inputs to the light source device 11 related to the delivery of the PBMT via a wired connection and/or a wireless connection. The PBMT can be applied to the skeletal muscle by a light signal that is generated by a light source device 11. To facilitate the delivery of the light signal to the skeletal muscle, the light source device 11 can be shaped so that at least a portion makes contact with the subject's skin proximal to the skeletal muscle.

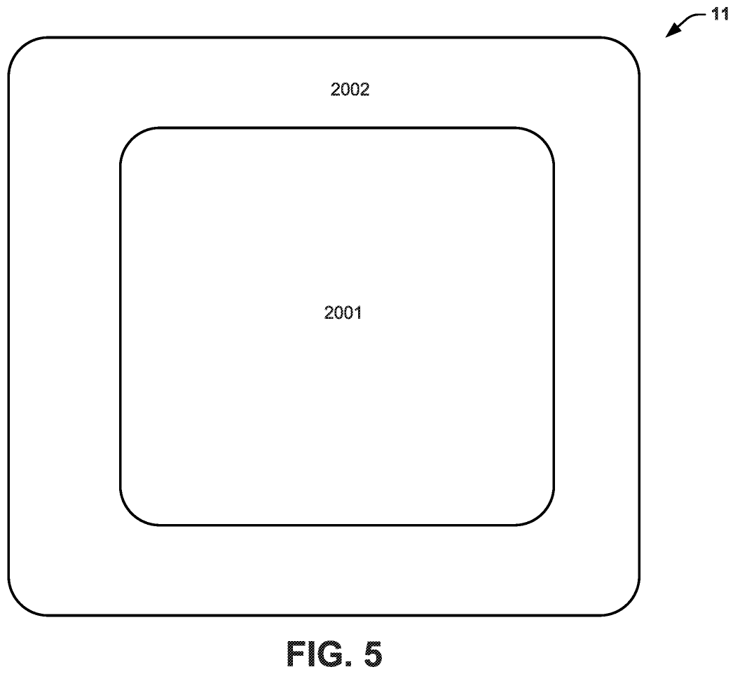

The light source device 11 can be configured in any shape that facilitates contacting a portion of the skin and/or the delivery of the light signal. An example of the light source device 11, including an electronics housing 2001 and a device housing 2002, is shown in FIG. 5. The electronics housing 2001 can include processing unit 14 and the power source and other electronics required for operation of the light source device 11. The device housing 2002 can surround the electronics housing and stabilize the electronics housing 2001. In some instances, the device housing 2002 can embody a securing mechanism to removably secure the light source device 11 to an area of the subject's skin. For example, the securing mechanism can be able to be disconnected to facilitate movement of the light source device 11. Even in the absence of the securing mechanism, the light source device 11 can be portable with at least a portion being able to be moved to different areas of the subject's body. Light delivery source clusters 13 can be within the electronics housing 2001 and/or within the device housing 2002.

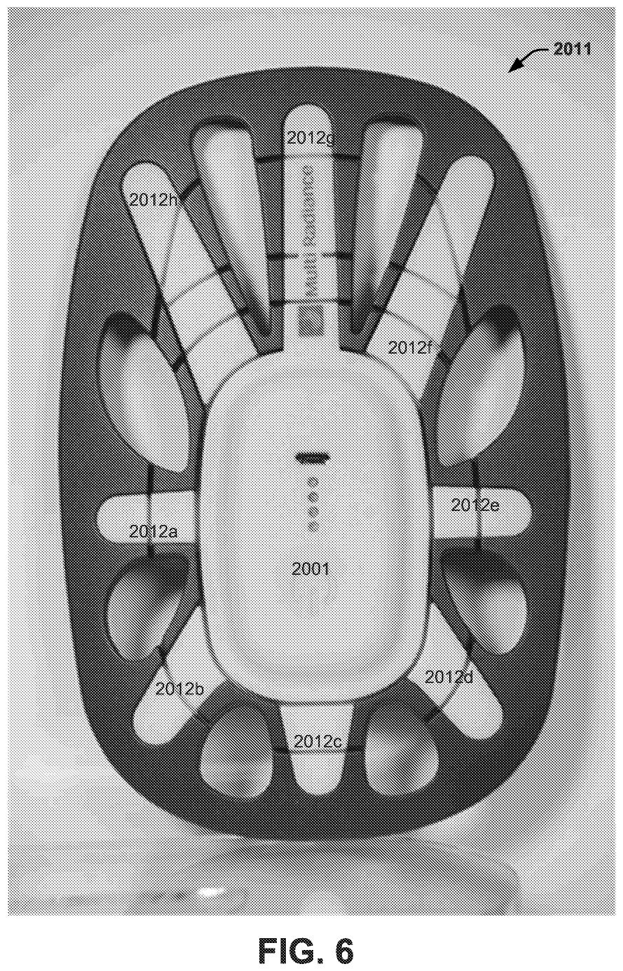

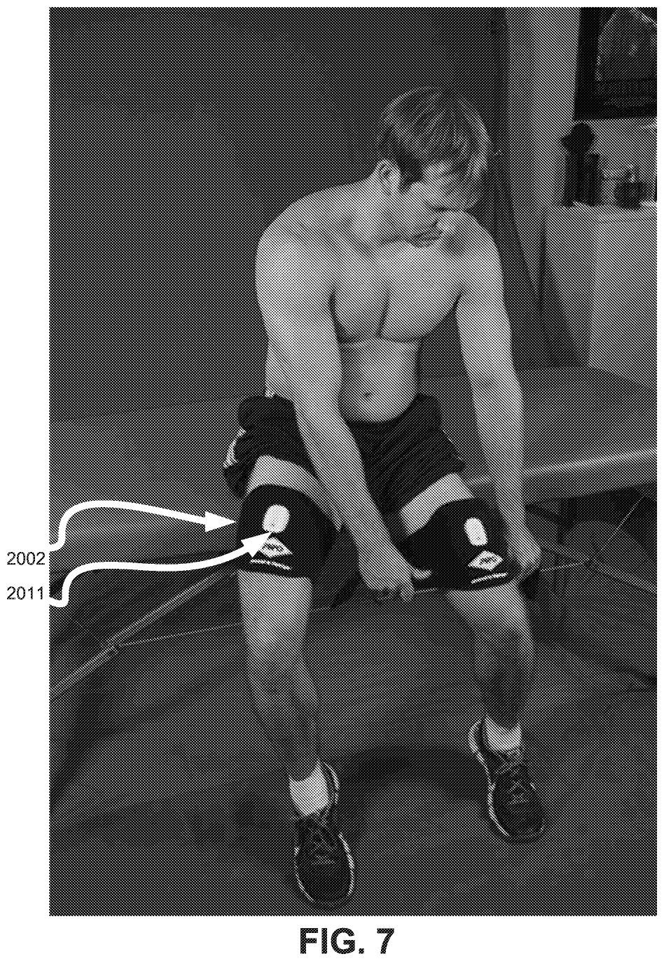

As one example, the light source device 11 can be embodied as an insert 2011 (shown in FIG. 6). The insert can include the electronics housing 2001 and a number of flanges 2012a-h extending from the device housing. Any number of flanges 2012a-h may exist, from 0 to N, where N is an integer limited only by the size of the insert. The electronics housing 2001 and/or the flanges can be made of a hard material (e.g., plastic or other hard material) and/or a flexible material (e.g., silicone, rubber, neoprene, or other flexible material) and configured with a shape or flexible into a shape that conforms to the target skeletal muscle. The insert can be inserted into a device housing 2002 as shown in FIG. 7. The device housing 2002 can be made of a flexible material (e.g., neoprene) and secured around an area of the subject's body that includes the skeletal muscle.

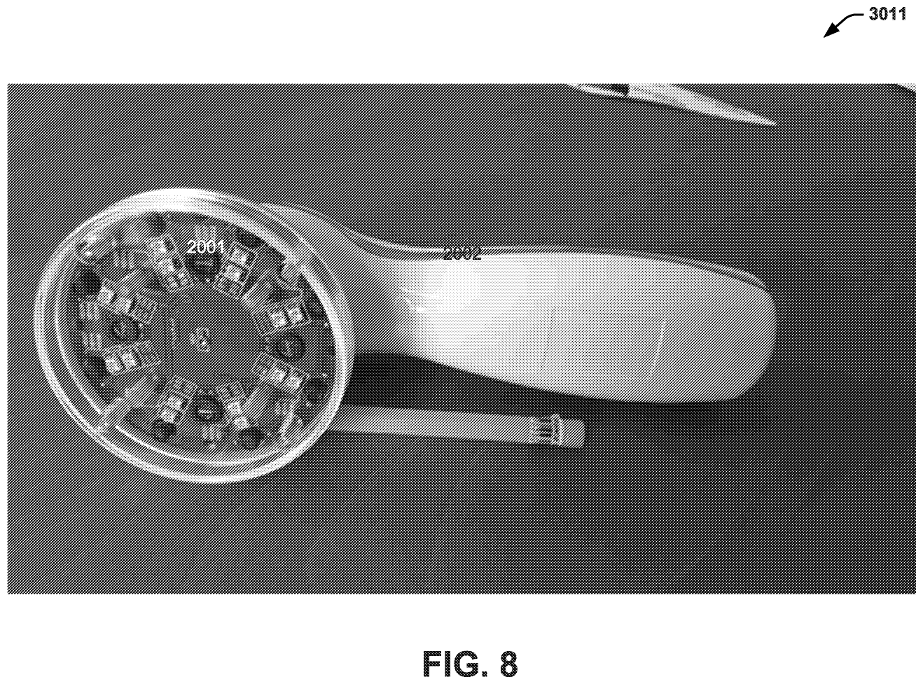

As another example, the light source device 11 can be embodied as a probe device 3011 (FIG. 8). The probe device 3011 can include a device housing 22 that is made of a hard material (e.g., a plastic) and include a portion configured to contact the subject's skin proximal to the skeletal muscle at a 90-degree angle to deliver the light signal. The electronics housing 2001 can be housed within the device housing 2002 with at least the light delivery source clusters 13 being included in an area that contacts the skin. Another example, although not illustrated, can include a flexible array device with a portion shaped to contact the skin at a 180-degree angle to deliver the light signal.

The light source device 11 can include at least one light delivery source to generate the light signal at a certain wavelength, with a certain power, in an operating mode. The operating mode can be at least one of a pulsed operating mode, a continuous operating mode, and a super-pulsed operating mode. The light source device 11 can also include a processing unit 14 programmed (e.g., preprogrammed, programmed in response to an input from the controller 12 (which may be in response to an input), or the like) with a time for application of the light signal to the skeletal muscle (e.g., the time can be sufficient to stimulate the phototherapeutic response in the skeletal muscle or a nerve associated with the skeletal muscle). The processing unit 14 can also be programmed with the certain wavelength, the certain power, and/or the operating mode. In some instances, the light source device 11 can also include a permanent magnet to provide a static (or constant) magnetic field, which can be used to secure the light source device 11 to the area of the subject's skin and/or to affect the light signal. The constant magnetic field can be from 5 mT to 1 T. Additionally, the light source device 11 can also include a power source. The power source, in some instances, can be an internal battery. In other instances, the power source can receive and/or store power from an external source. In some instances, the external source can be associated with the controller 12.

In some instances, the light signal can include a light wave at a single wavelength of light delivered in a certain operating mode. However, in other instances, the light signal can include a combination of a plurality of individual light waves with different wavelengths of light delivered in two or more different operating modes. The combination of individual light waves is advantageous because the individual light waves can work constructively to create a synergistic effect, enhancing each individual wavelength's ability to penetrate the skin, allowing for a greater portion of the available light energy to reach biological targets beneath the surface of the skin. Accordingly, the light signal can be configured so that individual light waves (from chosen light sources, with a selected wavelength, with a given power, and the like) within the light signal work constructively to create a synergistic effect.

The plurality of individual light waves can be generated by a plurality of light delivery sources. Accordingly, the light source device 11 can include a plurality of light delivery sources, each configured to deliver light of a certain wavelength, with a given power, in a pulsed operating mode, a continuous operating mode, or a super-pulsed operating mode. The light signal can be delivered by a light source device 11 that includes a combination of one or more super pulsed lasers (which deliver a desired peak power from an ultrashort pulse with a minimized level of heat accumulated in the patient's tissue), one or more infrared emitting diodes, and one or more light emitting diodes. In some instances, the light source device 11 can include groups of a super pulsed laser, an infrared emitting diode, and a light emitting diode. In other instances, the light source device can include groups of a super pulsed laser, at least three infrared emitting diodes, and at least three light source devices. The use of a super pulsed source can minimize the photo-thermal effect accumulating within the skin surface and target tissue.

One organization of the plurality of light delivery sources is in one or more light delivery source clusters 13 (an example of an individual cluster is shown in FIG. 2). In practice, the light source device can have any number of light delivery source clusters 13, limited only by the size of the area of the light source device 11 designated for delivery of the light signal.

As shown in FIG. 2, each light delivery source cluster 13 includes three types of light sources (LS1 16, LS2 17, LS3 15). However, the light delivery source clusters 13 may include a greater or fewer number of light sources. Three light sources are shown for simplicity of illustration and explanation. The light sources (LS1 16, LS2 17, LS3 15) each generate light waves with wavelengths within a wavelength range of 400-1100 nm. More particularly, LS1 16 can be configured to generate a first portion of the light signal with a wavelength from 600-680 nm; LS2 17 can be configured to generate a second portion of the light signal with a wavelength from 850-900 nm, and LS3 15 can be configured to generate a third portion of the light signal with a wavelength from 880-930 nm. In this example, LS3 15, which is in the middle of each light delivery source cluster 13, can operate in the super-pulsed operating mode, while LS1 16 and LS2 17, which surround LS3, can each operate in the continuous operating mode or the pulsed operating mode. In other words, LS3 can be a super pulsed laser that creates an impulse of high intensity that emits for a billionth of a second in synchrony with LS1 (like a red LED or a red light) and/or LS2 (an infrared source, like an infrared LED or an infrared light). Advantageously, the use of the super-pulsed laser (LS3) allows a desired peak power to be delivered for an ultrashort pulse with a minimized level of heat accumulated in the subject's skin and skeletal muscle (in other words, minimizes the photothermal effect).

Many configurations of each light delivery source cluster 13 are possible. Two examples of possible configurations are set forth, but countless other possibilities exist (including with other light sources), as long as there are one or more L1, one or more L2, one or more L3. One possible configuration of each light delivery source cluster 13 is a 1:1:1 configuration, with L3 (the super-pulsed laser) between L2 (the red source) and L3 (the infrared source). Another possible configuration of each light delivery source cluster 13 is a 1:3:3 configuration with L2 surrounded by three (or more) L2 and three (or more) L1. For example, in this configuration, L2 and L1 can alternate as they are arranged around L3 (e.g., L2 L1 L2 L1 L2 L1 surrounding L3). As another example, L2 and L1 can be grouped together around L3 (e.g., L2 L2 L2 L1 L1 L1). Although not expressly described, other example configurations are possible in the 1:3:3 light delivery source cluster 13. The light delivery source clusters 13 within the same light source device 11 can be configured identically, but need not have identical configurations. For example, a light source device 11 can have three light delivery source clusters, with one a 1:1:1 configuration and the other two 1:3:3 configurations. Additionally, clusters of L1, L2, and/or L3 alone and/or with one other of L1, L2, and L3 are possible.

### V. Methods

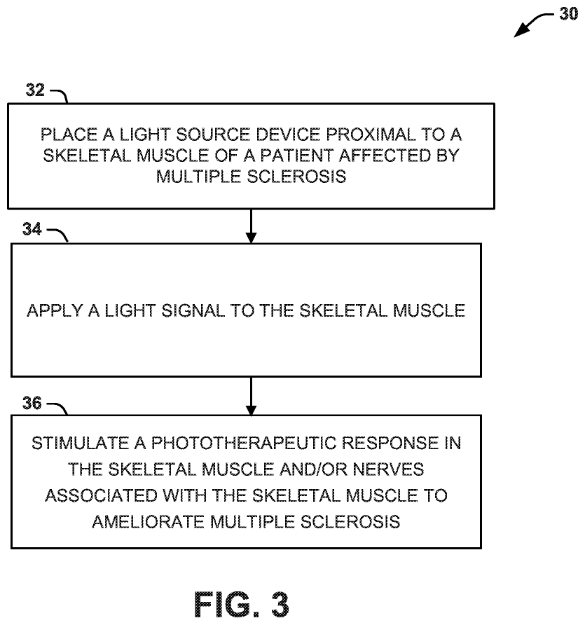

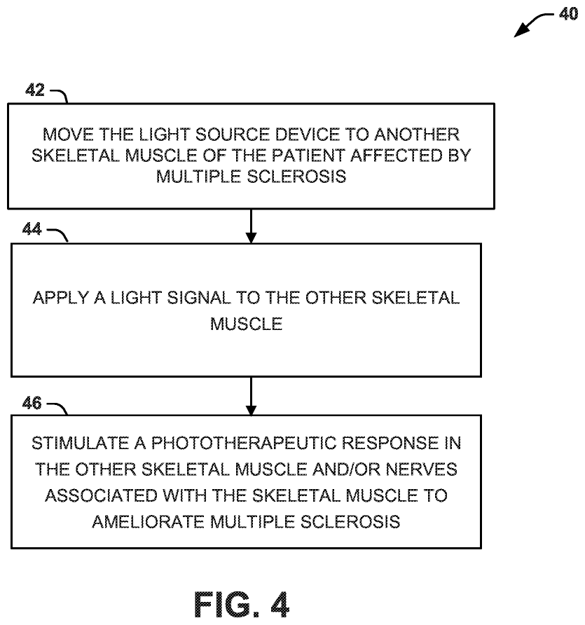

Another aspect of the present disclosure can include methods 30, 40 (FIGS. 3 and 4) for reducing fatigue in patients affected by multiple sclerosis (MS). FIG. 3 illustrates a method 30 for applying PBMT to a skeletal muscle of a patient affected by MS to ameliorate MS. FIG. 4 illustrates a method 40 for applying PBMT to another skeletal muscle of the patient affected by MS to further ameliorate MS. The methods 30, 40 can be executed by hardware for example, at least a portion of the system 10 shown in FIG. 1 and described above. Additionally, PBMT can be used alone or in combination with a traditional exercise therapy to ameliorate MS.

The methods 30 and 40 are illustrated as process flow diagrams with flowchart illustrations. For purposes of simplicity, the methods 30 and 40 shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 30 and 40. Additionally, one or more elements that implement the methods 30 and 40, such as light source device 11 and/or controller 12 of FIG. 1, may include a non-transitory memory and one or more processors that can facilitate the configuration and generation of the light signal.

Referring now to FIG. 3, shows a method 30 for applying PBMT to a skeletal muscle of a patient affected by MS to ameliorate MS. At step 32, a light source device (e.g. light source device 11) can be contacted to a subject's skin proximal to (e.g., directly adjacent or over) a skeletal muscle of a patient. The patient can be any patient who has been diagnosed with MS or who is symptomatic of MS.

At step 34, a light signal can be applied to the skeletal muscle. The light signal can be generated in at least one of a pulsed operating mode, a continuous operating mode, and a super-pulsed operating mode. The light signal can include one wave of a single wavelength. However, alternatively, the light signal can include a plurality of individual waves with multiple wavelengths. The combination of the plurality of individual waves can work constructively to create a synergistic effect, enhancing each individual wavelength's ability to penetrate the skin, allowing for a greater portion of the available light energy to reach biological targets beneath the surface of the skin. The light signal is applied for a time sufficient to stimulate a phototherapeutic response in nerves associated with the skeletal muscle and/or the skeletal muscle itself. At step 36, a phototherapeutic response can be stimulated in the nerves associated with the skeletal muscle and/or the skeletal muscle. The phototherapeutic response can include improving mitochondrial oxidative capacity due to modulation of mitochondrial cytochrome c-oxidase (CCO). As a result, the fatigue associated with MS can be ameliorated.

The method 30 continues in FIG. 4, which shows a method 40 that occurs after moving the light source device. At step 42, the light source device (e.g. light source device 11) can be moved to another area of the subject's skin proximal to another skeletal muscle. At step 44, a light signal can be applied to the other skeletal muscle. The light signal is applied for a time sufficient to stimulate a phototherapeutic response in nerves associated with the other skeletal muscle and/or the other skeletal muscle itself. At step 46, a phototherapeutic response can be stimulated in the other skeletal muscle and/or the other skeletal muscle itself, further ameliorating MS.

From the above description, those skilled in the art will perceive improvements, changes and modifications. Such improvements, changes and modifications are within the skill of one in the art and are intended to be covered by the appended claims. All patents, patent applications, and publications cited herein are incorporated by reference in their entirety.

1. A method comprising:
placing a light source device proximal to a skeletal muscle of a patient affected by multiple sclerosis; and
applying a light signal in at least one of a pulsed operating mode, a continuous operating mode, and a super-pulsed operating mode through the light source device to the skeletal muscle of the patient affected by multiple sclerosis, wherein the light signal is applied for a time sufficient to stimulate a phototherapeutic response in the skeletal muscle or a nerve associated with the skeletal muscle of the patient affected by multiple sclerosis
wherein the light source device comprises at least three light sources each configured to apply a portion of the light signal comprising a different wavelength within a wavelength range of 400-1100 nm,
wherein each of the at least three light sources operates in the pulsed operating mode, the continuous operating mode, or the super-pulsed operating mode.

2. The method of claim 1, wherein the light source device is a probe device or a flexible array device placed over the skeletal muscle of the patient affected by multiple sclerosis.

3. (canceled)

4. The method of claim 1, wherein the at least three light sources comprise:
a first light emitting diode configured to emit visible light in the pulsed operating mode or the continuous operating mode;
a second light emitting diode configured to emit infrared light in the pulsed operating mode or the continuous operating mode; and
a superpulsed laser configured to emit visible light or infrared light in the superpulsed operating mode.

5. The method of claim 4, wherein
the first light emitting diode is configured to emit light at a wavelength between 600 and 680 nm;
the second light emitting diode is configured to emit light at a wavelength between 850 and 900 nm; and
the superpulsed laser is configured to emit light at a wavelength between 880 and 930 nm.

6. The method of claim 4, wherein the light source device comprises:
at least three first light emitting diodes;
at least three second light emitting diodes; and
a superpulsed laser.

7. The method of claim 4, wherein the light source device comprises one or more clusters of at least one first light emitting diode, at least one second light emitting diode, and a superpulsed laser.

8. The method of claim 1, wherein the light source device further comprises a permanent magnet that provides a constant magnetic field from 5 mT to 1 T.

9. The method of claim 1, further comprising:
moving the light source device to another area of the patient;
placing the light source device over another skeletal muscle in the other area of the patient; and
applying the light signal through the light source device to the other skeletal muscle for a time sufficient to stimulate a phototherapeutic response in the other skeletal muscle or other nerve associated with the other skeletal muscle.

10. The method of claim 1, wherein the placing further comprises contacting the light source device to skin of the patient affected by multiple sclerosis over the skeletal muscle of the patient affected by multiple sclerosis.

11. A light source device configured to be placed proximal to a muscle of a patient affected by multiple sclerosis comprising:
at least one light source configured to apply a light signal in at least one of a pulsed operating mode, a continuous operating mode, and a super-pulsed operating mode to the skeletal muscle of the patient affected by multiple sclerosis;
a permanent magnet that provides a constant magnetic field from 5 mT to 1 T;
a processing unit preprogrammed with a time for application of the light signal, wherein the time is sufficient to stimulate a phototherapeutic response in the skeletal muscle or a nerve associated with the skeletal muscle of the patient affected by multiple sclerosis.

12. The light source device of claim 11, wherein the time is sufficient to deliver a total dose of at least 0.4 J/cm² to the skeletal muscle

13. (canceled)

14. The light source device of claim 11, wherein the at least one light source comprises at least three light sources each configured to apply a portion of the light signal comprising a different wavelength within a wavelength range of 400-1100 nm,
wherein each of the at least three light sources operates in the pulsed operating mode, the continuous operating mode, or the super-pulsed operating mode.

15. The light source device of claim 14, wherein the at least three light sources comprise:
a first light emitting diode configured to emit visible light in the pulsed operating mode or the continuous operating mode;
a second light emitting diode configured to emit infrared light in the pulsed operating mode or the continuous operating mode; and
a superpulsed laser configured to emit visible light or infrared light in the superpulsed operating mode.

16. The light source device of claim 15, wherein:
the first light emitting diode is configured to emit light at a wavelength between 600 and 680 nm;
the second light emitting diode is configured to emit light at a wavelength between 850 and 900 nm; and
the superpulsed laser is configured to emit light at a wavelength between 880 and 930 nm.

17. The light source device of claim 11, further comprising a housing, wherein the housing forms the light source device as a probe device or a flexible array device.

18. The light source device of claim 11, further comprising a securing mechanism to removeably secure the light source device to the patient's body.

19. The light source device of claim 11, further comprising a power source.

20. The light source device of claim 19, wherein the power source is at least one of a battery and a plug.
